(19) United States
(12) Patent Application Publication
(10) Pub. No.: US 2012/0178695 A1
(43) Pub. Date: Jul. 12, 2012

(54) METHODS OF TREATING NEUROPATHIC PAIN

(76) Inventor: Joseph Moskal, Evanston, IL (US)

(21) Appl. No.: 13/380,702

(22) PCT Filed: Jul. 2, 2010
(86) PCT No.: PCT/US2010/040905

§ 371 Date: Mar. 15, 2012

Related U.S. Application Data
(60) Provisional application No. 61/222,657, filed on Jul. 2, 2009.

(51) Int. Cl. A61K 38/07; A61P 29/00; A61P 25/00; A61K 38/02

(52) U.S. Cl. 514/17.3; 514/18.3

(57) ABSTRACT

The disclosure relates, at least in part, to methods of treating neuropathic pain in a patient in need thereof by administering an effective amount of a disclosed compound, e.g. a peptide NMDA receptor partial agonist.

### RELATED APPLICATIONS

This application claims priority to U.S. Ser. No. 61/222,657 filed Jul. 2, 2009, hereby incorporated by reference in its entirety.

### BACKGROUND

Medications from several different drug classes have been used to treat neuropathic pain, including tricyclic antidepressants, anticonvulsants, topical agents, and opioid and non-opioid analgesics. Such treatment regimes currently available for neuropathic pain (a leading cause of chronic pain) at best, approximately 30% are effective in significantly diminishing the pain, and may lose their efficacy over time. Although numerous pharmacological agents are available for the treatment of neuropathic pain, a definitive therapy has remained elusive.

The most common side-effect of the non-opiate analgesics is sedation or somnolence. Based on data from the package inserts for these drugs, as many as 20-30% of patients experience sedation. Significant and persistent sedation can pose other risks, including locomotor function impairment that can lead to falling and the inability to perform many daily functions such as driving. With opioids, when administered over prolonged periods, undesirable side effects such as drug tolerance, chemical dependency and even physiological addiction can occur.

Competitive receptor antagonists have been shown to alleviate neuropathic pain in preclinical studies, but were ineffective in clinical trials [e.g., 3₁₃ (2_carboxypoperazin-4-yl)propyl-lphosphonic acid] Kristensen, J D et al., Pain, 1991, 51, 249-253.) Noncompetitive ion channel blockers have not proved to be of therapeutic value, as they typically show unacceptable psychotomimetic side effects (e.g. MK.-801) (Leung A. et al., Pain, 2005, 91, 177-187.) Glycine B binding site antagonists do not show psychotomimetic side effects, but typically induce ataxia and 5 sedation along with being poor ‘blood-brain barrier crossers’ (Id.). Recently developed NR₂B-specific noncompetitive receptor antagonists, such as traxoprodil (Nakazato et al., Pharmacol., 2005, 73, 8-14), have shown promise, but side effects such as dizziness and depression have also been reported. Finally, gabapentin (Neurontin) also has been reported to cause sedation, ataxia, and dizziness (Gilron I., Curr Opin Anaesthesiol, 2007, 20, 456-472).

Duration of action is also a limitation for most of the leading therapies. This is particularly important as neuropathic pain, can lead to other factors (e.g. insomnia and/or depression) that impact the patient's overall quality of life. Therefore, achieving pain relief with a sufficient duration is an important factor for neuropathic pain drugs. There remains a need for better treatments of neuropathic pain and associated pain disorders and/or conditions with compounds that can provide improved efficacy and/or reduced undesirable side effects.

### SUMMARY

A method for treating neuropathic pain in a patient in need thereof, comprising administering to said patient a pharmaceutically effective amount of a NMDA receptor modulating peptide, is provided herein. For example, the present invention is directed in part to a method for treating neuropathic pain by administering a therapeutically effective dose of GLYX-13, as disclosed herein, or derivative thereof, for example, a peptide having NMDA receptor partial agonist activity, e.g., a peptide that binds the glycine site of a NMDA receptor. For example, provided herein is a method for treating neuropathic pain in a patient in need thereof, comprising administering to said patient a pharmaceutically effective amount of a peptide that modulates a glycine site on a NMDA receptor.

In some embodiments, neuropathic pain is associated with a condition selected from the group consisting of herpes, HIV, traumatic nerve injury, stroke, post-ischemia, fibromyalgia, reflex sympathetic dystrophy, complex regional pain syndrome, spinal cord injury, sciatica, phantom limb pain, diabetic neuropathy, and cancer chemotherapeutic-induced neuropathic pain.

For example, in some embodiments, a contemplated peptide that is capable of modulating the glycine site of a NMDA receptor is represented by (GLYX-13):

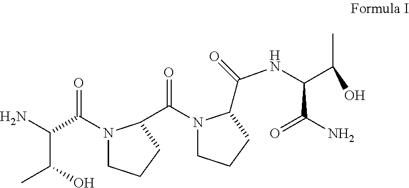

or pharmaceutically acceptable salts thereof.

In some embodiments, a contemplated peptide is administered intravenously, intraperitoneally, intramuscularly, or subcutaneously, for example, a contemplated method may include administering a single-dose of said peptide. In some embodiments, disclosed methods may provide, after about 1 day, or even after 8 days of administration of a disclosed peptide, substantial improvement in neuropathic pain of a patient. In another embodiment, administration of disclosed peptides does not result in significant axatia in the patient.

In some embodiments, disclosed peptides or compounds may be administered daily. A pharmaceutically effective amount of a disclosed peptide or compound may be about 0.01 mg/kg to about 1000 mg/kg.

Also provided herein is a method of treating neuropathic pain in a patient in need thereof, comprising administering to said patient a single dose of a compound represented by:

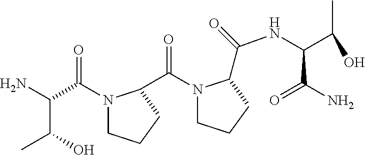

or pharmaceutically acceptable salts thereof, wherein after 1 day, or after 8 days, the patient has substantial improvement in neuropathic pain. A single dose may include for example, about 0.01 mg/kg to about 1000 mg/kg of a disclosed peptide.

In another embodiment, the disclosed invention relates to administering a dipyrrolidine peptide compound comprising the sequence Thr-Pro-Pro-Thr, or exemplified by Formula I (GLYX-13) for the treatment of neuropathic pain in mammals including humans.

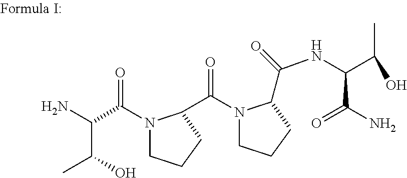

### BRIEF DESCRIPTION OF THE DRAWINGS

FIG. 1 depicts the antinociceptive actions of GLYX-13 and gabapentin in the rat formalin model of tonic pain. Percentage analgesia is the percentage reduction (on the basis of the area under the curve for vehicle+formalin) in flinches in the late phase response (10-40 min) after intraplantar formalin injection (50 ml of 5% formalin) compared with control values arbitrarily set at 100%. Drugs were administered subcutaneously, 15 min before formalin. N=8-10 per group. Mean±SEM.

FIG. 2 depicts the result of groups of eight male rats subjected to chronic constriction injury to the right hind limb. Following development of peripheral neuropathy, rats were administered GLYX-13, i.v. at 5, 10 or 20 mg/kg on days 12 and 13 post-operative (PO). Rats were tested for mechanical allodynia at 15 and 60 minutes post-dose on day 13 PO. The data represent mechanical allodynia readings at 15 minutes post-dose.

FIG. 3 depicts the results of groups of 12 male rats subjected to chronic constriction injury to the right hind limb. Following development of peripheral neuropathy, rats were 5 administered GLYX-13, i.v. at 5, 10 or 20 mg/kg on days 12 and 13 post-operative (PO). Rats were tested for mechanical allodynia at 15 and 60 minutes post-dose on day 13 PO. The data represent mechanical allodynia readings at 60 minutes post-dose.

FIG. 4 depicts mean±SEM percent analgesia in the late phase (30-50 min) in 3 month old Sprague-Dawley rats pretreated with GLYX-13 (3 mg/kg i.v.), ketamine (10 mg/kg i.v.) or saline vehicle injection (1 mg/ml i.v. tail vein) in freely behaving rats 8 days before left rear paw intraplantar injections (50 μl) of formalin (1.5%). Analgesia is % reduction in licking time, number of flinches, or total pain score (favoring+2×foot up+3×licking time). The vehicle group exhibited 311.0 sec of mean licking, 18.4 number of mean flinches, and 2876.7 mean pain score. N=10-11 per group. * P<0.05 Fisher PLSD post hoc drug vs. all other groups.

### DETAILED DESCRIPTION

Exemplary peptides contemplated for use in the disclosed methods are illustrated below. In an embodiment, a contemplated peptide is a tetrapeptide having the amino acid sequence Thr-Pro-Pro-Thr, L-threonyl-L-prolyl-L-prolyl-L-threonine amide. Also contemplated are any suitable salt forms such as, but not limited to, the acetate salt. Contemplated peptides may be cyclized or non-cyclized form as for example, further described in U.S. Pat. No. 5,763,393. Glycine-site partial agonists of the NMDA receptor are disclosed in U.S. Pat. No. 5,763,393, U.S. Pat. No. 6,107,271, and Wood et al, NeuroReport, 19, 1059-1061, 2008, the entire contents of which are herein incorporated by reference.

Representative contemplated peptides include the following peptides listed below; contemplated peptides may be obtained by well-known recombinant or synthetic methods such as those described in U.S. Pat. Nos. 5,763,393 and 4,086,196 herein incorporated by reference.

| | |
|---|---|
| NT-1: SEQ ID. NO: 1. | Lys--Ala--Ser--Gln--Asp--Val--Ser--Thr--Thr--Val-Ala |
| NT-2: SEQ ID. NO: 2. | Ser--Ala--Ser--Tyr--Arg--Tyr--Thr |
| NT-3: SEQ ID. NO: 3. | Gln--Gln--His--Tyr--Ser--Thr--Pro--Pro--Thr |
| NT-4: SEQ ID. NO: 4. | Val--Tyr--Tyr--Ser--Gln--Gln--His--Tyr--Ser--Thr--Pro--Pro-Thr |
| NT-5: SEQ ID. NO: 5. | Glu--Asp--Leu--Ala--Val--Tyr--Tyr--Ser--Gln--Gln--His--Tyr--Ser--Thr-- |
| | Pro--Pro--Thr |
| NT-6: SEQ ID. NO: 6. | Ser--Val--Gln--Ala--Glu--Leu--Asp--Leu--Ala--Val--Tyr--Tyr--Ser--Gln-- |
| | Gln--His--Tyr--Ser--Thr--Pro--Pro--Thr |
| NT-7: SEQ ID. NO: 7. | Phe--Thr--Ile--Ser--Ser--Val--Gln--Ala--Glu--Leu--Asp--Leu--Ala--Val-- |
| | Tyr--Tyr--Ser--Gln--Gln--His--Tyr--Ser--Thr--Pro--Pro--Thr |
| NT-8: SEQ ID. NO: 8. | Gln--Gln--His--Tyr--Ser--Thr--Pro--Pro--Thr--Phe--Gly--Gly--Gly |
| NT-9: SEQ ID. NO: 9. | Gln--Gln--His--Tyr--Ser--Thr--Pro--Pro--Thr--Phe--Gly--Gly--Gly--Thr-- |
| | Lys--Leu--Glu |
| NT-10: SEQ ID. NO: 10: | 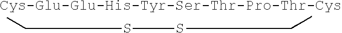 |
| NT-11: SEQ ID. NO: 11 | Ser--Gln--Gln--His--Tyr--Ser--Thr--Pro--Pro--Thr-Ser |
| NT-12: SEQ ID. NO: 12 | Gln--Gln--His--Tyr--Ser |
| NT-13: SEQ ID. NO: 13 | Thr--Pro--Pro--Thr |
| NT-14: SEQ ID. NO: 14 | Thr--Pro--Pro |
| NT-15: SEQ ID. NO: 15 | Pro--Pro--Thr |
| NT-16: SEQ ID. NO: 16 | Pro--Pro |
| NT-17: SEQ ID. NO: 17 | Thr--Pro--Thr |
| NT-18: SEQ ID. NO: 18 | Thr |

Neuropathic pain typically results from damage to or dysfunction of the peripheral or central nervous system, rather than stimulation of pain receptors. Diagnosis may be, for example, suggested by pain out of proportion to tissue injury, dysesthesia (e.g., burning, tingling), and signs of nerve injury detected during neurologic examination. Neuropathic pain can also result from the administration of chemotherapeutics such as paclitaxol.

Syndromes associated with neuropathic pain contemplated for treatment herein include but are not limited to postherpetic neuralgia, root avulsions, painful traumatic mononeuropathy, painful polyneuropathy (particularly due to diabetes), central pain syndromes (potentially caused by virtually any lesion at any level of the nervous system), postsurgical pain syndromes (e.g., postmastectomy syndrome, postthoracotomy syndrome, phantom pain), and complex regional pain syndrome (reflex sympathetic dystrophy and causalgia). For example, contemplated herein are methods for treating neuropathic pain associated with spinal cord injury, HIV, traumatic nerve injury, spinal cord injury, sciatica, herpes—e.g., post-herpetic neuralgia, diabetic neuropathy, phantom limb pain, stump/neuroma pain, post-ischemic pain (stroke), fibromyalgia, reflex sympathetic dystrophy (RSD), complex regional pain syndrome (CRPS), cancer-chemotherapeutic induced neuropathic pain, vertebral disk rupture, and/or trigeminal neuralgia.

In some embodiments, contemplated methods relate to use of a disclosed peptide or peptides alone or in combination with one or more other pain-reducing agents for manufacturing a medicament for treating acute neuropathic pain including provide relief from such pain. In a preferred embodiment, the disclosure relates to methods for treating neuropathic pain by administering an effective amount of GLYX-13 to a patient in need thereof. GLYX-13, as defined herein (Formula I) is a partial agonist at the glycine site of the NMDA receptor complex. At low doses, GLYX-13 can activate this receptor, whereas at higher does, GLYX-13 behaves as a receptor antagonist. One basis for GLYX-13's pharmacological activity against neuropathic pain relates to its ability to act as a weak antagonist at the glycine site of the NMDA receptor complex. Neuropathic pain and/or a variety of neuropathic pain conditions may be treated according to a disclosed method without significantly affecting behavior or motor coordination, and/or without significantly inducing or promoting seizure activity.

For example, in a disclosed method, a contemplated peptide, e.g., GLYX-13, or a composition comprising a contemplated peptide and e.g., a pharmaceutically acceptable excipient, may be administered parenterally to a patient including but not limited to subcutaneously and intravenously. The compound or compositions of the invention may also be administered via slow controlled i.v. infusion or by release from an implant device. In an embodiment, a disclosed method for treating neuropathic pain includes administering one dose, or one or more doses, of a disclosed peptide. In some embodiments, a patient has substantial improvement in neuropathic pain after 12 hours, after 1 day, after 1 week, after 2 days, after 3 days, after 4 days, after 5 days, after 6 days, or even after 8 days of a one (single) dose administration.

A therapeutically effective amount of a disclosed peptide required for use in therapy varies with the nature of the pain condition being treated, the length of treatment time desired, the age and the condition of the patient, and is ultimately determined by the attending physician. In general, however, doses employed for adult human treatment typically are in the range of about 0.01 mg/kg to about 1000 mg/kg per day. The dose may be about 1 mg/kg to about 100 mg/kg per day. The desired dose may be conveniently administered in a single dose, or as multiple doses administered at appropriate intervals, for example as two, three, four or more sub-doses per day.

A number of factors may lead to the compounds (peptides) of a disclosed invention being administered over a wide range of dosages. When given in combination with other therapeutic agents, the dosage of the compounds of the present invention may be given at relatively lower dosages. As a result, the dosage of a compound of the present invention 5 may be from about 1 ng/kg to about 100 mg/kg. The dosage of a compound of the present invention may be at any dosage including, but not limited to, about 1 ug/kg, 25 ug/kg, 50 ug/kg, 75 ug/kg, 100 u ug/kg, 125 ug/kg, 150 ug/kg, 175 ug/kg, 200 ug/kg, 225 ug/kg, 250 ug/kg, 275 ug/kg, 300 ug/kg, 325 ug/kg, 350 ug/kg, 375 ug/kg, 400 ug/kg, 425 ug/kg, 450 ug/kg, 475 ug/kg, 500 ug/kg, 525 ug/kg, 550 ug/kg, 575 ug/kg, 600 ug/kg, 625 ug/kg, 650 ug/kg, 675 ug/kg, 700 ug/kg, 725 ug/kg, 750 ug/kg, 775 ug/kg, 800 ug/kg, 825 ug/kg, 850 ug/kg, 875 ug/kg, 900 ug/kg, 925 ug/kg, 950 ug/kg, 975 ug/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg.

Disclosed peptides may be provided as part of a liquid or solid formulation, for example, aqueous or oily suspensions, solutions, emulsions, syrups, and/or elixirs. The compositions may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl hydroxybenzoate and sorbic acid. Contemplated compounds/peptides may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

### EXAMPLES

### Example 1

### Antinociceptive Action of GLYX-13 in Rats

Antinociceptive actions of GLYX-13 were conducted in a rat formalin assay. Male Sprague-Dawley rats (125-170 g) were manually restrained for a subcutaneous injection of 1.5% formalin (5 ml with a 26 ga needle) into the lateral footpad on the plantar surface of the left hind paw. After formalin injections, rats were placed in individual clear plastic cylinders of 30 cm diameter. Drug effects on the second phase of the pain response were monitored with observations conducted over the period between 10 and 40 min after formalin injection. Each group was composed of 10 animals. Vehicle, GLYX-13, or gabapentin was administered subcutaneously at the nape of the neck 10 min before the formalin injection. The time spent licking or elevating the injected limb was quantitated over this 20 min observation period.

Next, GLYX-13 was evaluated in the chronic constriction nerve injury model of neuropathic pain; 5 mg/kg GLYX-13. Male Sprague-Dawley rats (200-225 g) were anesthetized with sodium pentobarbitone (6 mg/kg, intraperitoneally) and supplemented as necessary with isoflurane (1-3% in oxygen). Under asceptic conditions, the right sciatic nerve was exposed by blunt dissection at the mid thigh level and 1 cm freed of adhering connective tissue. Four chromic catgut (4.0) ligatures were tied to lightly constrict the nerve at 1 mm intervals. The overlying muscle and skin were sutured and, upon recovery from anesthesia, the rats returned to cages of soft padded bedding and to cages with sawdust bedding after 24 h. At days 5, 7, 9, and 11 post recovery, mechanical anodynia was assessed with Von Frey filaments (calibration numbers, 3.61-6.10) applied to the plantar surface of the hind paw from below. The filaments were evaluated in ascending order with the threshold for both the ipsilateral and contralateral paws being evaluated. The withdrawal threshold was defined as the lowest force of two or more consecutive Von Frey filaments to elicit a withdrawal reflex. Only animals that developed mechanical allodynia (withdrawal response r5g of force) in their nerve-injured paw by day 11 were utilized for drug testing.

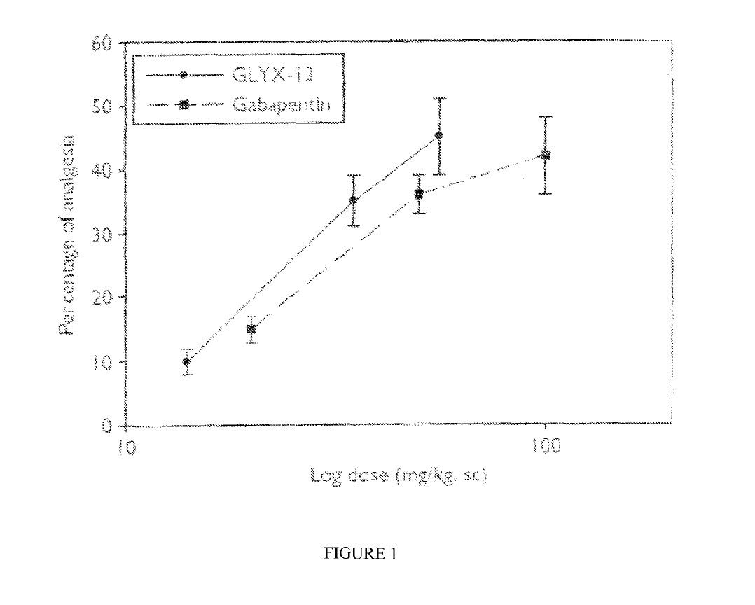

Both GLYX-13 and gabapentin demonstrated dose-dependent efficacy in the rat formalin model of tonic pain (FIG. 1). Gabapentin-treated rats were ataxic at the highest dose, whereas GLYX-13-treated rats were not ataxic at any of the doses examined (data not shown). No statistically significant differences in analgesic effects of GLYX-13 and gabapentin were observed.

The antinociceptive actions of vehicle or GLYX-13 (5 mg/kg) were evaluated at 15 and 60 minutes after dosing. Unlike the study shown in FIG. 1, these studies were performed intravenously. GLYX-13 was antinociceptive at both time points with no evidence of ataxia. Ataxia was further evaluated utilizing a rotor-rod apparatus and GLYX-13 was not found to induce ataxia at 30 min after 5, 50, or 500 mg/kg intravenous administration. No data have been collected past the 60 min time point. Mechanical allodynia is modulated by central rather than peripheral mechanisms. GLYX-13 readily crosses the blood-brain barrier in an active form.

GLYX-13 demonstrated significant antinociceptive activity in the rat formalin model of tonic pain and in the rat constriction nerve injury model of neuropathic pain at doses not induce ataxia, in contrast to e.g. gabapentin. The results with GLYX-13 show that NMDA receptor glycine-site partial agonists may be excellent therapeutic candidates for the treatment of neuropathic pain.

### Example 2

### Acetic Acid Writhing Study in Mice

GLYX-13 was tested for antinociceptive activity in an acetic-acid induced writhing model in mice. Groups of 10 male mice were dosed i.v. with either saline or GLYX-13 at 1, 5, or 10 mg/kg. Five minutes later, each mouse was injected i.p. with a solution of 0.5% acetic acid in 0.9% saline and observed for writhing behavior for five minutes. The total number of writhes for each mouse was recorded and the mean number of writhes was compared between the control and GLYX-13 treated groups. Intravenous administration of GLYX-13 at doses of 1, 5, and 10 mg/kg did not block acetic acid induced writhing behavior in male mice.

### Example 3

### Radiant Heat Tail Flick Study in Rats

The ability of GLYX-13 to block the radiant heat tail flick response was determined in male rats. Groups of 10 male rats were administered either saline or GLYX-13 at 1, 5, or 10 mg/kg via i.v. injection, and five minutes following administration, the tail of each rat was exposed to a radiant heat stimulus. The time to elicit a characteristic tail flick was determined for each rat and the mean response time was determined for each group. Administration of GLYX-13 to male rats at doses of 1, 5, and 10 mg/kg did not result in a change in the response time to a radiant heat stimulus, suggesting that GLYX-13 is not directly analgesic against thermal pain in rats. Administration of GLYX-13 at doses of 5, 10, and 20 mg/kg to male rats exhibiting unilateral peripheral neuropathy caused a marked increase in the withdrawal threshold in the nerve injured hind paw to mechanical allodynia.

### Example 4

### Randall-Selitto Assay in Rats

The purpose of this study was to assess the potential effect of GLYX-13 upon the pain threshold in rats using the Randall-Selitto paw pressure model. Groups of 10 male rats (20 for saline vehicle control) were injected with a 20% suspension of yeast in the right hind paw and two hours later, each rat was intravenously administered either saline or GLYX-13 at 1, 5, or 10 mg/kg. The response to mechanical pain stimulus was determined for both the inflamed and non-inflamed paw using an analgesia meter. The amount of force required to elicit paw withdrawal was measured for each rat. GLYX-13 at does of 1, 5, or 10 mg/kg had no significant effect on the pain threshold in rats. Slight, but not statistically significant, increases in the pain threshold were observed in the rats does with 5 and 10 mg/kg.

### Example 5

### Neuropathic Pain Study in Rats

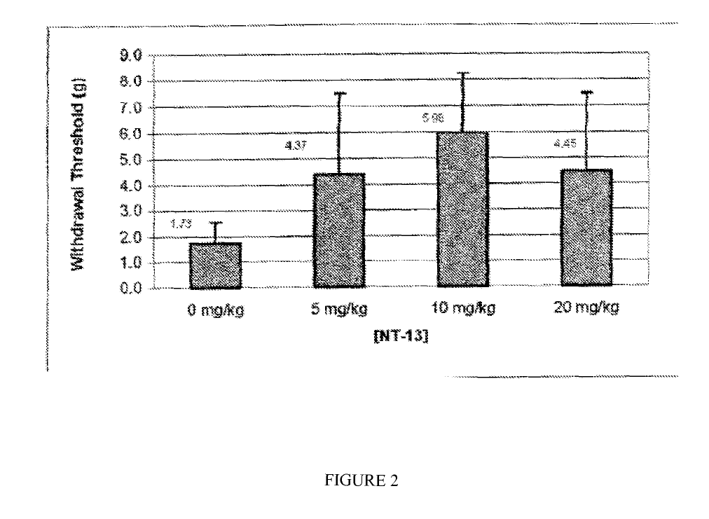

The ability of GLYX-13 to attenuate neuropathic pain was assessed in a rat model of peripheral neuropathy. In this study, four groups of eight male rats were subjected to surgically-induced peripheral neuropathy. Peripheral neuropathy was induced in the right hind limb of male rats by loose ligation of the right sciatic nerve according to the method of Bennett and Xie (Bennett and Xie, 1988). After 8-10 days, a peripheral neuropathy had developed in the right hind limbs as determined by sensitivity to both mechanical allodynia and thermal hyperalgesia. This pilot study consisted of four groups of eight male rats. Days 12-13 post surgery, vehicle (0.9% saline) or GLYX-13 at 5, 10, and 20 mg/kg were administered, and rats were tested for sensitivity to both the thermal stimulus and mechanical allodynia. Administration of GLYX-13 at doses of 5, 10, and 20 mg/kg to males rats exhibiting unilateral peripheral neuropathy caused a marked increase in the withdrawal threshold in the nerve injured hind paw to mechanical allodynia. The results for GLYX-13 15 minutes post dose in this rodent neuropathy model are shown FIG. 2.

GLYX-13, at a dose of 10 mg/kg elicited withdrawal thresholds of 5.98±2.25 g and 7.26±2.19 g at 15 and 60 minutes, respectively relative to control values of 1.73±0.82 and 5 9.01±3.95 g. Doses of 5 and 20 mg/kg GLYX-13 also caused small but distinct increases in withdrawal threshold. At approximately 60 min post-dose, the group mean withdrawal threshold was still raised (5.04±3.09 g) in the low dose group, however the high dose threshold had begun to return towards the pre-dose value (2.08±1.21 g). No marked changes were observed in the responses of the uninjured (contralateral) hind paw at any of the time points tested. Intravenous administration of GLYX-13 (at doses of 5, 10, and 20 mg/kg, free base) to neuropathic rats caused a slight reduction in the sensitivity of the nerve-injured paw to mechanical stimuli (Von Frey filaments), at approximately 15 and 60 min post dose. The 10 mg/kg dose (10 mg/kg, free base) appeared to be most effective, with the most marked increase in the withdrawal threshold of the nerve-injured paw, at both time points.

### Example 6

### Neuropathic Pain Study in Rats

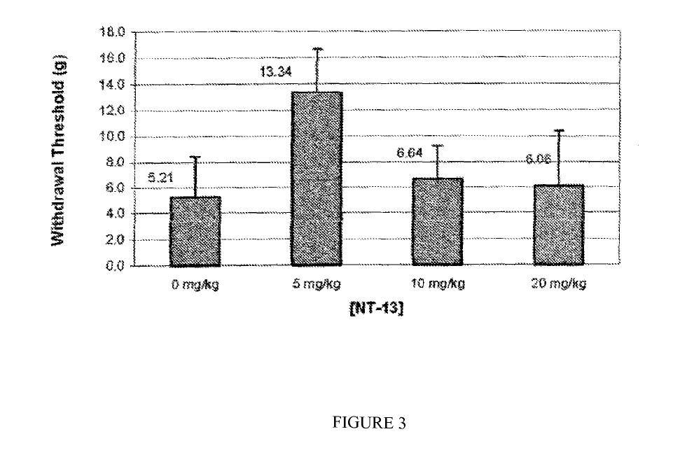

A second peripheral neuropathy study was performed with GLYX-13 in male Sprague- Dawley rats. A peripheral mononeuropathy was induced in the right hind limb and rats were tested for development of mechanical allodynia. Rats that had positively developed neuropathy were administered vehicle (0.9% saline) or GLYX-13 i.v. at doses of 5, 10, or 20 mg/kg on days 12 and 13 PO. Testing for mechanical allodynia was performed at 15 and 60 minutes post-dose. Intravenous administration of GLYX-13 (5 mg/kg, free-base) to neuropathic rats caused a statistically significant reduction in the sensitivity of the nerve-injured paw to mechanical stimuli (Von Frey filaments) at approximately 15 and 60 min post dose, with no evidence of any contralateral effects. The effect of GLYX-13 on mechanical allodynia observed at 60 minutes post dose are shown in FIG. 3. In this rodent peripheral neuropathy model, i.v. administration of5 mg/kg GLYX-13 to male rats caused a significant reduction in the sensitivity of the nerve injured hind paw to mechanical allodynia at both 15 and 60 minutes post dose. There was no evidence of any systemic or contralateral effects in this study. The 5 mg/kg dose was the most effective. These results would suggest that even low doses of GLYX-13 can protect rats against painful neuropathy, and that these effects can last up to an hour post-dosing The present data suggest that intravenous GLYX-13 can alleviate painful neuropathy in rodents for up to an hour post dose, and this effect is consistent with the known ability of NMDA receptor antagonists to relieve neuropathic pain.

### Example 7

### Neuropathic Pain 8 Day Test

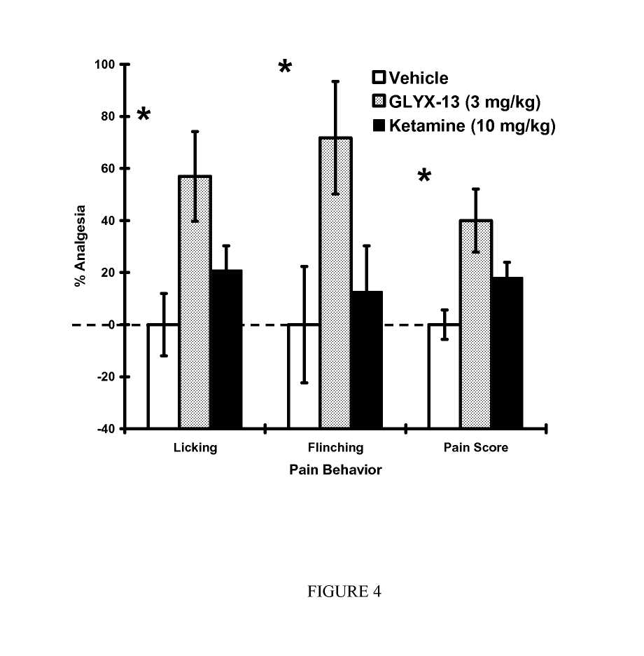

Freely behaving 3 month old Sprague Dawley rats were pretreated with GLYX -13 (3 mg/kg i.v.), ketamine (10 mg/kg i.v.) or saline vehicle injection (1 mg/ml i.v. tail vein) 8 days before left rear paw intraplantar injections (50 μl) of formalin (1.5%). FIG. 4 indicates that rats receiving 1 dose of GLYX-13 exhibited increased analgesia 8 days after administration, and shows superiority to ketamine in the same model.

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The entire contents of all patents, published patent applications, websites, and other references cited herein are hereby expressly incorporated herein in their entireties by reference.

What is claimed is:

1. A method for treating neuropathic pain in a patient in need thereof, comprising administering to said patient a pharmaceutically effective amount of a peptide that modulates a glycine site on a NMDA receptor.

2. The method of claim 1, wherein the neuropathic pain is associated with a condition selected from the group consisting of herpes, HIV, traumatic nerve injury, stroke, postischemia, fibromyalgia, reflex sympathetic dystrophy, complex regional pain syndrome, spinal cord injury, sciatica, phantom limb pain, diabetic neuropathy, and cancer chemotherapeutic-induced neuropathic pain.

3. The method of claim 1, wherein the NMDA receptor modulating peptide is represented by: 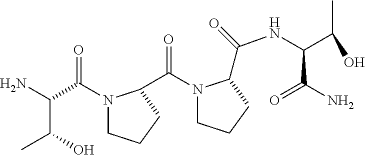
or pharmaceutically acceptable salts thereof.

4. The method of claim 1, wherein the peptide is administered intravenously, intraperitoneally, intramuscularly, or subcutaneously.

5. The method of claim 1, wherein the method comprises administering a single-dose of said peptide.

6. The method of claim 1, wherein about 1 day after administration the patient has substantial improvement in neuropathic pain.

7. The method of claim 1, wherein about 8 days after administration the patient has substantial improvement in neuropathic pain.

8. The method of claim 1, wherein the compound is administered daily.

9. The method of claim 1, wherein upon said administration, the patient has no significant axatia.

10. The method of claim 1, wherein the pharmaceutically effective amount is about 0.01 mg/kg to about 1000 mg/kg.

11. A method of treating neuropathic pain in a patient in need thereof, comprising administering to said patient a single dose of a compound represented by: 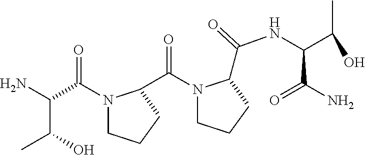
or pharmaceutically acceptable salts thereof, wherein after 1 day the patient has substantial improvement in neuropathic pain.

12. The method of claim 11, wherein after 8 days the patient has substantial improvement in neuropathic pain.

13. The method of claim 11, wherein the single dose comprises about 0.01 mg/kg to about 1000 mg/kg.

14. A method of treating neuropathic pain in a patient in need thereof, comprising administering to said patient an effective amount of SEQ ID. NO: 13 (Thr--Pro--Pro-Thr).


SEQUENCE LISTING (19 sequences)

SEQ ID NO: 1 - protein, 11 aa, Artificial Sequence
  Description of Artificial Sequence Synthetic peptide
KASQDVSTTVA

SEQ ID NO: 2 - protein, 7 aa, Artificial Sequence
  Description of Artificial Sequence Synthetic peptide
SASYRYT

SEQ ID NO: 3 - protein, 9 aa, Artificial Sequence
  Description of Artificial Sequence Synthetic peptide
QQHYSTPPT

SEQ ID NO: 4 - protein, 13 aa, Artificial Sequence
  Description of Artificial Sequence Synthetic peptide
VYYSQQHYSTPPT

SEQ ID NO: 5 - protein, 17 aa, Artificial Sequence
  Description of Artificial Sequence Synthetic peptide
EDLAVYYSQQHYSTPPT

SEQ ID NO: 6 - protein, 22 aa, Artificial Sequence
  Description of Artificial Sequence Synthetic peptide
SVQAELDLAVYYSQQHYSTPPT

SEQ ID NO: 7 - protein, 26 aa, Artificial Sequence
  Description of Artificial Sequence Synthetic peptide
FTISSVQAELDLAVYYSQQHYSTPPT

SEQ ID NO: 8 - protein, 13 aa, Artificial Sequence
  Description of Artificial Sequence Synthetic peptide
QQHYSTPPTFGGG

SEQ ID NO: 9 - protein, 17 aa, Artificial Sequence
  Description of Artificial Sequence Synthetic peptide
QQHYSTPPTFGGGTKLE

SEQ ID NO: 10 - protein, 11 aa, Artificial Sequence
  Description of Artificial Sequence Synthetic peptide
CEEHYSTPPTC

SEQ ID NO: 11 - protein, 11 aa, Artificial Sequence
  Description of Artificial Sequence Synthetic peptide
SQQHYSTPPTS

SEQ ID NO: 12 - protein, 5 aa, Artificial Sequence
  Description of Artificial Sequence Synthetic peptide
QQHYS

SEQ ID NO: 13 - protein, 4 aa, Artificial Sequence
  Description of Artificial Sequence Synthetic peptide
TPPT

SEQ ID NO: 14 - protein, 3 aa, Artificial Sequence
  Description of Artificial Sequence Synthetic peptide
TPP

SEQ ID NO: 15 - protein, 3 aa, Artificial Sequence
  Description of Artificial Sequence Synthetic peptide
PPT

SEQ ID NO: 16 - protein, 2 aa, Artificial Sequence
  Description of Artificial Sequence Synthetic peptide
PP

SEQ ID NO: 17 - protein, 3 aa, Artificial Sequence
  Description of Artificial Sequence Synthetic peptide
TPT

SEQ ID NO: 18 - protein, 1 aa, Artificial Sequence
  Description of Artificial Sequence Synthetic peptide
T

SEQ ID NO: 19 - protein, 4 aa, Artificial Sequence
  Description of Artificial Sequence Synthetic peptide
TPPT